(19) United States
(12) Patent Application Publication
(10) Pub. No.: US 2004/0024003 A1
(43) Pub. Date: Feb. 5, 2004

(54) Flat medicinal preparation for transmucosal administration of oxycodon or a comparable active ingredient in the oral cavity, for use in pain therapy and in addiction therapy

(76) Inventors: Bodo Asmussen, Bendorf (DE); Markus Krumme, Neuwied (DE)

Correspondence Address: BIRCH STEWART KOLASCH & BIRCH, PO BOX 747, FALLS CHURCH, VA, 22040-0747, US

(21) Appl. No.: 10/149,980

(22) PCT Filed: Dec. 1, 2000
(86) PCT No.: PCT/EP00/12093

(30) Foreign Application Priority Data
Dec. 14, 1999 (DE) 199 60 154.2

(51) Int. Cl. A61K 31/485; A61K 9/70

(52) U.S. Cl. 514/282; 424/443

(57) ABSTRACT

A flat pharmaceutical preparation which is able to disintegrate in aqueous media and is in the form of a sheet, film, paper or wafer for transmucosal administration of active ingredients in the oral cavity, is characterized by a content of oxycodone, or an active ingredient comparable to oxycodone, or a therapeutically suitable salt of oxycodone or of the pharmacologically comparable active ingredient.

The present invention relates to a flat pharmaceutical preparation for transmucosal administration of oxycodone or a pharmacologically comparable active ingredient in the region of the oral cavity, and to the use of such a pharmaceutical preparation in pain therapy and in replacement therapy for the treatment of opiate and cocaine dependence. The invention particularly relates to flat pharmaceutical preparations of the type mentioned which are able to disintegrate in aqueous media and are in the form of sheets, films, papers or wafers. The invention also embraces processes suitable for the production of such pharmaceutical preparations.

The use of opiates in pain therapy makes special demands on the dosage forms employed for this purpose. The main difficulty concerns adjusting the dose for the pain experienced subjectively by each individual patient, that is to say according to requirements. In this connection the aim is to avoid both exposure of the patient to unreasonable states of pain and the development of tolerance and possibly dependence as a result of overdosage. For this reason, it is desirable and necessary for suitable active ingredients and dosage forms which make a rapid onset of the analgesic effect possible to be available in order if necessary to make a rapid dose adjustment which is needed. For this reason, the active ingredient and the dosage form should ensure entry into the blood in a time which is as short as possible. In addition, such a dosage form should allow self-administration by the patient in an uncomplicated and, at the same time, reliable manner.

Conventional dosage forms such as, for example, tablets which disintegrate in the stomach and release the active ingredient there are therefore less suitable because the effect usually has its onset only after a considerable d lay. Although this disadvantage is lessened with tablets which disintegrat even in the mouth and whose active ingredient is absorbed through the oral mucosa, it must be taken into account in this connection that a considerable proportion of the active ingredient preparation reaches the stomach with the saliva and therefore is unavailable for rapid absorption through the oral mucosa. In addition, gastrointestinal absorption is followed by a relatively rapid metabolic degradation of the active ingredient in the liver (first-pass effect).

For these and other reasons, flat dosage forms such as, for example, preparations in the form of films or wafers are advantageous. The small thickness by comparison with the area results in a short diffusion pathway when such a pharmaceutical form is applied, for example, to the oral mucosa. This leads to a rapid dissolution of the preparation under the action of saliva and to correspondingly rapid release of the active ingredient, which can be absorbed rapidly and directly through the oral mucosa.

Flat active ingredient carriers have already been developed and produced for various purposes. The basis for these dosage forms can be regarded as being provided by DE-A 27 46 414, which describes a sheet-like ribbon of active ingredient, binder and other excipients. In this case, because of the homogeneous thickness, density and width there is a direct connection between a unit length of the ribbon and the dose of active ingredient present therein. The advantages of the possibility of continuous dosing have also been recognized by other applicants and have been described in specific individual variants. Thus, German patent 36 30 603 describes a flat carrier material, for example in the form of a separating paper with an active ingredient-containing coating, it being possible to detach the latter dose-wise from the carrier material after previous division into dosage units.

DE-A 196 52 188 describes a flat pharmaceutical preparation which is suitable for the administration and release of the opiate analgesic buprenorphine in the oral cavity. However, with this dosag form a large part of th amount of active ingredient present therein is transported with the saliva into the stomach and metabolized, because this dosage form has insufficient or nonexistent muco-adhesiveness.

It is true that the general advantages of flat dosage forms are known in the prior art, for example the previously mentioned more rapid delivery of active ingredient and possibility of simpler dosing, also the possibility of discrete intake, that is to say without the assistance of liquid, also advantages in the production, and the possibility of printing during production, whereby the reliability of intake can be increased.

Despite the described advantages, such flat dosage forms have scarcely been accepted to date. Presumably, many manufacturers of pharmaceuticals estimate the benefits as too slight by comparison with conventional dosage forms, so that it does not appear worthwhile to develop products of this type and seek approval for them under medicinal product legislation. An additional factor is that there would be a need for large investment because the machinery available and the existent know-how could not be utilized for these novel types of products. The costs of such a changeover evidently do not appear to be justified because the therapeutic or economic benefits of these flat dosage forms is usually categorized as not great enough by comparison with conventional pharmaceutical forms such as, for example, tablets. Especially when the active ingredient can be administered orally in any event, the cost of developing an alternative dosage form is a deterrent, even if the advantages associated therewith are known.

The analgesic active ingredients very suitable for peroral administration include the opiate oxycodone which has been employed successfully for many years in pain therapy. Following peroral administration, two thirds of it are bioavailable, that is to say it appears in the bloodstream to a very effective extent.

How ver, the precondition for transmucosal, for example buccal or sublingual, administration in the oral cavity is that the oral mucosa displays adequate permeability for the active ingredient, taking account of the necessary dose. The permeability in turn depends to a large extent on the physicochemical properties of the active ingredient. Oxycodone is effective even in small amounts and displays good peroral absorption, the average duration of action being 4-6 h. The times taken to enter the blood with normal peroral administration are 15-30 min. This time is too long for adjusting the dose to requirements and means that the patient has to wait an unnecessarily long time until the onset of alleviation.

The object of the invention was therefore to provide pharmaceutical preparations based on and having the general advantages of flat active ingredient carriers, which have, through the combination with a specific active ingredient, additional therapeutic and/or economic advantages compared with pharmaceutical preparations of the same active ingredient based on conventional dosage forms. It is moreover intended that the said active ingredient be released in the oral cavity in such a way that the disadvantages described in the prior art do not occur. In particular, the object was to provide an administration form for oxycodone which releases the active ingredient in the oral cavity without having the disadvantages described in the prior art. The pharmaceutical forms are furthermore intended to be at the same time safe and simple to use and to meet the practical requirements of pain therapy or addiction-cessation therapy. The object of the invention was further to indicate processes for producing such preparations, which make production possible under competitive conditions. The object is surprisingly achieved by a flat pharmaceutical preparation which is able to disintegrate in aqueous media and is in the form of a sheet, film, paper or wafer and which has a content of oxycodone, or an active ingredient comparabl to oxycodone, or a therapeutically suitable salt of oxycodone or of the pharmacologically comparable active ingredient.

A novel pharmaceutical preparation according to claim 1 is, as is explained hereinafter, far superior to a conventional dosage form for administering oxycodone, both from the economic and from the therapeutic viewpoint, and is particularly suitable on the one hand for analgesia for states of severe pain, but on the other hand for the therapy of opiate or cocaine dependence in the form of a replacement therapy or of an abstinence-achieving programme.

The pharmaceutical preparation according to claim 1 can on administration be brought into direct contact with the oral mucosa. The flat configuration results immediately after the administration in at least approximately one half of the surface area, which is anyway large, of the dosage form being directly located on the mucosa. The oxycodone released from the preparation thus finds two factors which are particularly favourable for entry into the body, namely a short diffusion distance and a large diffusion area.

Even with the simplest configuration of the invention—where the disintegration time is a few minutes after administration or after introduction into an aqueous medium—the superiority of an oxycodone-containing film compared with an oxycodone-containing tablet will thus be shown. The advantageous properties of the novel preparations appear so distinctly because oxycodone is effective even in low doses. The present invention combines the great efficacy of oxycodone with the advantageous release and delivery characteristics of flat transmucosal dosage forms. This means that the invention makes available pharmaceutical preparations which are able to make a highly effective analgesic available in the body in an efficient and rapid manner. In this connection, the invention makes use of the fact that the oral mucosa displays, because of the physicochemical characteristics of oxycodon, good permeability for this active ingredient, which is why the latter is particularly suitable for buccal or sublingual administration.

The only short time delay between administration of the novel pharmaceutical preparation and the uptake in the body means that the patient experiences alleviation of rapid onset and he is able if required to add further dose units of the pharmaceutical preparation in order thus to increase the dose stepwise—as required—so that an inappropriately high dosage or overdosage can be avoided. It is thus possible to a certain extent to “titrate” as required the sensations of pain occurring. Such a procedure is also appropriate for avoiding the development of tolerance.

The novel pharmaceutical preparation is preferably used for transmucosal administration of oxycodone or its pharmaceutically acceptable salts or other pharmacologically acceptable derivatives of oxycodone. Although oxycodone—where appropriate in the form of one of its therapeutically acceptable salts—is the most preferred active ingredient, the invention also includes active ingredients which are pharmacologically similar or comparable to oxycodone, because the described advantages of the invention may also apply thereto, although to a different extent. Further suitable active ingredients “which are pharmacologically similar or comparable to oxycodone” mean, in particular, those which are to be counted among the opiates or opioides, because many of them display not only pharmacodynamic but also pharmacokinetic similarities to oxycodone, that is to say activity at relatively low dose, great ability to cross membranes and high first-pass effect. From this group, particular preference is given as active ingredients to derivatives of morphine or dihydromorphine, and substances from the methadone and fentanyl groups.

With the novel preparations, delivery of active ingredient takes place by permeation through the oral mucosa. The precondition for this is that the flat preparation is in close contact with the mucosa during th administration period, that is to say if possible until the preparation has dissolved or disintegrated. It is possible by choosing suitable excipients to improve contact of the novel pharmaceutical preparation with the oral mucosa. For this reason, the pharmaceutical preparation contains in a preferred embodiment of the invention an adhesion-promoting excipient or an excipient mixture which confers bioadhesive or mucoadhesive properties on the preparation. It is known of certain excipients which can be administered orally and are used in pharmacy that they have mucosa-adherent properties. Examples of such mucoadhesive substances are polyacrylic acid, carboxymethylcellulose, hydroxymethylcellulose, methylcellulose, tragacanth, alginic acid, gelatin and gum arabic. It is additionally known of various non-mucoadhesive substances that in certain mixing ratios they likewise display mucoadhesive properties. One example of such a mixture is glycerol monooleate/water in the ratio 84:16 (Engström et al., Pharm. Tech. Eur. 7[1995], No. 2, pp. 14-17).

On use of bioadhesive or mucoadhesive excipients, preference is to be given to a bilayer or multilayer structure of the dosage form of the novel preparation. It is possible, by providing only the layer or layers facing the oral mucosa or in contact therewith with a mucoadhesive finish, but not the layer or layers located distally or outwardly, to avoid the preparation sticking different areas of mucosa together during the period of use, which would lead to considerably unpleasant sensations during use. Preferred embodiments therefore have a bilayer or multilayer structure, with one of the two layers or, in the case of a multilayer structure, one of the layers having bioadhesive or mucoadhesive properties.

In embodiments which contain non-mucoadhesive layers in addition to mucoadhesive ones, the former are preferably designed so that their permeability for the active ingredient is lower than that of the bioadhesive or mucoadhesive layer. This makes it possibl to avoid active ingredient being released in the saliva in the oral cavity, which would lead to losses of active ingredient.

The present invention also embraces preparations which, in addition to oxycodone or a comparable active ingredient, contain at least one other active ingredient for transmucosal administration. A preparation of this type may be advantageous in several respects. On the one hand, it is an acknowledged method for treating a plurality of symptoms or states occurring simultaneously by administering a fixed combination of active ingredients in a single medicine. For this purpose it is possible to incorporate any therapeutically worthwhile active ingredients into the novel preparation.

On the other hand, the combination, provided in another embodiment of the invention, of an opiate active ingredient with another substance which is able to reduce the specific risks of opiate administration is particularly worthwhile and advantageous. Thus, for example, opiate antagonists—or else partial opiate antagonists—such as, for example, nalbuphine, naloxone, naltrexone or levallorphan can be combined with the opiate active ingredient, which results in the danger of addiction or habituation through repeated administration of the preparation being reduced by the fact that the dose cannot be increased without at the same time accepting an increase in the antagonistic effect. The success of such a strategy will depend essentially on the choice of a suitable antagonist and of the dose ratio in the preparation.

In order not to promote misuse or improper use, the novel pharmaceutical preparation will preferably be previously divided into doses and be present separate from one another in a suitable pack, so that for removal of a dose unit in each case the latter will be made removable, for example in the form of a blister pack in which each dose unit is sealed individually in a thermoformed well.

However, it may also be worthwhile in the framework of programmes for treating opiate or cocaine dependence for example to offer the managing physicians the preparation in the form of pack units in which it is present in the form of undivided sheet- or ribbon-like material, from which the dose units can be separated for the purpose of administration. This facilitates mass administration and gives the administering physicians the same possibility of detaching different dose units from one and the same material depending on the dose required.

Since the extent of the bioavailability to be expected from the novel pharmaceutical preparation is greater than that for known preparations, it is necessary where appropriate to adjust the dose. In the case of oxycodone, the analgesic single dose will be 5 to 20 mg, but for use in addiction therapy or replacement therapy it may be distinctly higher.

The production of the pharmaceutical preparations takes place according to the invention in several steps. Two basic process variants are suitable for producing a starting material in web form, from which finally either the single doses or else whole pack units are separated by cutting or punching. The first group of processes comprises those in which a ribbon or a processing sheet is uniformly coated with aqueous or solvent-containing liquids which may in some cases have an increased viscosity, and is subsequently subjected to a drying process. For this purpose, initially the coating composition is produced, which requires intimate mixing of at least one water-soluble polymer capable of film formation, and of the active ingredient(s) and of a suitable vaporizable liquid. It is possible if necessary to incorporate further excipients such as disintegration-modifying polymers, bioadhesive or mucoadhesive excipients, plasticizers, fillers, texturizing substances, pigments, dyes, taste-masking agents, solubilizers, substances to adjust the pH, smoothing agents, flatting agents, disintegration promoters etc. An alternative possibility is to produce the starting material in w b form by thermoforming, that is to say without the assistance of liquids. This includes all hot-m lt coating processes and all extrusion processes. A precondition in this case is that the polymer or polymer mixture capable of film formation is thermoformable. The required ingredients are mixed and shaped under the action of pressure and/or heat by extrusion, blow-moulding or by coating ribbons or sheets and, after solidification, passed on for further processing.

Appropriately modified processes are suitable for producing novel preparations with a multilayer structure, it being immaterial whether a plurality of materials in web form are produced and joined together simultaneously or successively.

1. Flat pharmaceutical preparation which is able to disintegrate in aqueous media and is in the form of a sheet, film, paper or wafer for transmucosal administration of oxycodone or of a therapeutically suitable salt of oxycodone in the oral cavity, characterized in that it has a bilayer or multilayer structure, with one of the layers being given bioadhesive or mucoadhesive properties by addition of an adhesion-promoting excipient or excipient mixture, and with the non-bioadhesive or -mucoadhesive layer(s) having a permeability for the active ingredient which is lower than that of the bioadhesive or mucoadhesive layer.

2. Pharmaceutical preparation according to claim 1, characterized in that it contains at least one other active ingredient for transmucosal administration.

3. Pharmaceutical preparation according to claim 2, characterized in that the said other active ingredient is suitable for preventing, moderating or delaying a dependence on opiates.

4. Pharmaceutical preparation according to claim 3, characterized in that the said other active ingredient is able at least partly to act as opiate antagonist.

5. Pharmaceutical preparation according to claim 4, characterized in that the said other active ingredient is selected from the group comprising nalbuphine, naloxone, naltrexone and levallorphan.

6. Pharmaceutical preparation according to one or more of the preceding claims, characterized in that it is in the form of an undivided material in the form of a sheet or ribbon, from which dose units can be separated for the purpose of administration.

7. Pharmaceutical preparation according to one or more of the preceding claims, characterized in that it is in a form previously divided dose-wise.

8. Pharmaceutical preparation according to claim 6 or 7, characterized in that it has an active ingredient content per dose unit which is suitable for analgesia, preferably an active ingredient content of 5-20 mg per dose unit.

9. Pharmaceutical preparation according to claim 6 or 7, characterized in that it has an active ingredient content per dose unit which is suitable for opiate or cocaine replacement therapy.

10. Use of a pharmaceutical preparation according to one or more of claims 1 to 12 for the production of a medicinal product which can be administered orally for pain treatment.

11. Use of a pharmaceutical preparation according to one or more of claims 1 to 12 for the production of a medicinal product which can be administered orally for opiate or cocaine replacement therapy or abstinence-achieving therapy.

12. Method for the treatment of states of pain by administration of a pharmaceutical preparation according to one or more of claims 1 to 8 onto the oral mucosa.

13. Method for the treatment of opiate or cocaine dependence within the framework of an abstinence-achieving or replacement therapy by administration of a mucoadhesive pharmaceutical preparation onto the oral mucosa, with the pharmaceutical preparation containing oxycodone or one of its therapeutically suitable salts as active ingredient, and with said active ingredient being administered in a transmucosal manner.

14. M thod according to claim 13, characteriz d in that the pharmaceutical preparation used has a bilayer or multilayer structure, with one of the layers being given bioadhesive or mucoadhesive properties by addition of an adhesion-promoting excipient or excipient mixture, and with the non-bioadhesive or -mucoadhesive layer(s) having a permeability for the active ingredient which is lower than that of the bioadhesive or mucoadhesive layer.

15. Method according to claim 14, characterized in that the pharmaceutical preparation used is a preparation according to claims 2 to 9.
